(19) United States
(12) Patent Application Publication
(10) Pub. No.: US 2021/0011009 A1
(43) Pub. Date: Jan. 14, 2021

(54) METHOD AND APPARATUS FOR ANALYSING SKIN-PRINTS

(71) Applicant: INTELLIGENT FINGERPRINTING LIMITED, Cambridge ( Cambridgeshire) (GB)

(72) Inventors: Melanie Jane Bailey, Guildford (Surrey) (GB); Roger Paul Webb, Guildford (Surrey) (GB); Catia Costa, Guildford (Surrey) (GB)

(21) Appl. No.: 16/499,140

(22) PCT Filed: Mar. 27, 2018
(86) PCT No.: PCT/GB2018/000050

§ 371 Date: Sep. 27, 2019

(30) Foreign Application Priority Data
Mar. 30, 2017 (GB) 1705128.5

(51) Int. Cl. G01N 33/543; A61B 10/00; A61B 5/00; B01L 3/00; G01N 33/483; G01N 33/487; G01N 33/94

(52) U.S. Cl. CPC G01N 33/5438; A61B 10/0096; A61B 5/4266; A61B 5/4845; B01L 3/5023; B01L 3/508; B01L 2400/027; G01N 33/48714; G01N 33/94; B01L 2300/0654; B01L 2300/0822; B01L 2300/126; G01N 33/4833

(57) ABSTRACT

A method of analysing a skin-print comprising the steps of providing a porous substrate, the porous substrate extending in a substrate plane and being substantially planar and having a first end and a second end, wherein the second end tapers to a point. The method further comprises: applying to the porous substrate a skin-print to be analysed; applying a fluid to the porous substrate; applying a voltage between the first end of the porous substrate and ground to generate an electric field, by which droplets of the fluid are ionised and emitted from the point in the form of a Taylor cone; and analysing the ionised droplets using a mass spectrometer.

### TECHNICAL FIELD

The disclosure relates to analysis of a skinprint, such as a fingerprint.

### BACKGROUND

An impression left by the friction ridges of human skin, such as the skin of a human finger contains information regarding the identity of the human. It is widely known that the appearance of the impression of the human finger, known as a fingerprint, is unique to each human and may be used to confirm the identity of the human. The appearance of the impression of the skin of other human body parts may also be unique to each human and so may also be used to confirm the identity of the human. Such impressions of human skin, when not specific to the skin of the human finger, may be called skin-prints.

In addition to the appearance of the impression left by human skin, the impression may contain chemical species which themselves may be detected in order to obtain further information. Skin-prints (especially fingerprints) comprise eccrine sweat and may contain other constituents that may form a target for a diagnostic test.

For example, when a human intakes a substance (e.g. by ingestion, inhalation or injection) the substance may be metabolised by the human body giving rise to secondary substances known as metabolites. The presence of a particular metabolite can be indicative of a specific intake substance. The intake substance and/or metabolites may be present in sweat and, as such, may be left behind in the skin-print, e.g. a fingerprint. Detection of such metabolites in a skin-print can be used as a non-invasive method of testing for recent lifestyle activity such as (but not limited to) drug use, or compliance with a pharmaceutical or therapeutic treatment regime.

Importantly, the taking of a skin-print is much simpler than obtaining other body fluids such as blood, saliva and urine, and is more feasible in a wider range of situations. Moreover, skin-print samples do not constitute biohazards to the same degree as blood or urine samples. Furthermore, since the appearance of the skin-print itself provides confirmation of the identity of the person providing the skin-print, there can be greater certainty that the substance or substances in the skin-print are associated with the individual. This is because substitution of a skin-print, particularly a fingerprint, is immediately identifiable from appearance whereas substitution of, for example, urine, is not immediately identifiable from appearance. As such, testing for one or more substances in a skin-print provides a direct link between the one or more substances and the identity of the human providing the skin-print.

It is known to employ mass spectrometry techniques to identify the constituents of a sample. One such technique is known as paper spray mass spectrometry and involves depositing a sample on a porous, wicking substrate, typically a planar material such as paper. A voltage and a solution are applied to the substrate. An electric field arising from the voltage causes gas phase ions to be produced from the surface of the substrate. These ionic species, or neutral compounds, may then be detected via conventional mass spectrometry techniques. A mass spectrometer is used to measure mass-to-charge (m/z) of single molecules or atoms in their ionised state.

Conventional paper spray mass spectrometry is not suitable for detecting the types of analytes at the quantities and concentrations that might be expected in a skin-print such as a fingerprint.

Given that the area distribution of analyte that might be present in a skin-print is less than what might be present in a drop of blood or urine, in order for the technique to perform effectively for skin-prints, significant work has been undertaken, as described below, to provide appropriate arrangements for the detection of analytes in skin-prints via the technique.

### STATEMENTS OF INVENTION

Against this background, there is provided a mass spectrometry technique suitable for analysing a skin-print.

In a first aspect of the disclosure, there is provided a method of analysing a skin-print comprising the steps of:
providing a porous substrate, the porous substrate being substantially planar and having a first end and a second end, wherein the second end tapers to a point;
applying to the porous substrate a skin-print to be analysed;
applying a fluid to the porous substrate;
applying a voltage between the first end of the porous substrate and ground to generate an electric field by which droplets of the fluid are ionised and emitted from the point in the form of a Taylor cone;
analysing the ionised droplets using a mass spectrometer.

The porous substrate may be substantially triangular.

The first end may form a base of the triangular porous substrate and the second end may form an apex of the triangular porous substrate.

The mass spectrometer may comprise a mass spectrometer inlet having an aperture and an inlet axis substantially perpendicular to the aperture.

The mass spectrometer inlet may be located such that the inlet axis is substantially perpendicular to the point of the porous substrate.

The mass spectrometer inlet may be located such that the inlet axis is substantially in the substrate plane.

The mass spectrometer inlet may comprise a frusto-conical aperture wherein the inlet axis is substantially coincident with the axis of the frusto-conical aperture.

The area of the porous substrate may be between 0.2 cm² and 3.0 cm², preferably between 1.0 cm² and 2.0 cm², more preferably between 1.5 cm² and 1.9 cm², still more preferably between 1.6 cm² and 1.7 cm², most preferably approximately 1.68 cm².

The voltage may be between 2 kV and 10 kV, preferably between 3.5 kV and 5.5 kV, and more preferably, between 4.4 kV and 4.6 kV, most preferably 4.5 kV.

The step of applying the fluid to the porous substrate may comprise applying a volume of fluid of between 10 μl and 1,000 μl, preferably between 50 μl and 200 μl, more preferably between 70 μl and 130 μl, still more preferably between 90 μl and 110 μl, and most preferably approximately 100 μl.

The method may further comprising the step of:
performing an optical analysis of the skin-print prior to applying the fluid to the porous substrate.

The step of performing the optical analysis may comprise performing a colourmetric analysis of the skinprint.

The method may further comprise the step of:
applying a developing agent to the porous substrate including the skin-print to be analysed in order to develop the skinprint prior to applying the fluid to the porous substrate.

The developing agent may be or may comprise Ninhydrin.

The developing agent may be or may comprise silver nitrate.

The method may further comprise a step of developing the developing agent by application of electromagnetic radiation.

The step of developing the developing agent may comprise exposing the developing agent to ultra-violet light.

The ultra-violet light may have a wavelength of approximately 254 nm.

The step of exposing the developing agent to ultra-violet light may be conducted for a period of 1 to 15 minutes, preferably 2 to 10 minutes, more preferably 3 to 7 minutes, even more preferably 4 to 6 minutes and most preferably approximately 5 minutes.

The optical analysis may be performed on the skin-print following application of the developing agent.

The porous substrate may be treated with silver nitrate prior to applying the skin-print to be analysed to the porous substrate.

In a second aspect of the disclosure, there is provided a cartridge comprising a housing and a porous substrate located within the housing, the porous substrate being substantially planar and having a first end and a second end, wherein the second end tapers to a point.

The cartridge may further comprise a first electrical contact connected to a part of the porous substrate distant from the point and a second electrical contact on an exterior of the housing electrically connected to the first electrical contact.

The cartridge may further comprise a shutter openable to provide access to the porous substrate and closable to restrict access to the porous substrate.

The housing may further comprise at least a first part and a second part which are movable relative to one another

i) from a first closed configuration in which the substrate chamber is inaccessible;

ii) to a first open configuration in which access to the substrate chamber is enabled to allow capturing of a skin-print on a sample-capture substrate contained within the substrate chamber; and subsequently

iii) into a second closed configuration in which the substrate chamber is again inaccessible;
wherein the cartridge further comprises a retaining mechanism for retaining the cartridge in the second closed configuration wherein the retaining mechanism is disablable to permit movement of the cartridge out of the second closed configuration
wherein the cartridge further comprises a tamper evident feature that prevents access to the retaining mechanism,
wherein access to the retaining mechanism for disabling the retaining mechanism is available only by triggering the tamper evident feature. a first closed position,

Access to the retaining mechanism for disabling the retaining mechanism may be restricted.

The cartridge may further comprise a non-return feature which prevents movement from the first open configuration to the first closed configuration.

The cartridge may further comprise a second open configuration accessible only by disabling the retaining mechanism.

The tamper evident feature may comprise a frangible element having an undamaged initial configuration in which the retaining mechanism is inaccessible and a damaged configuration which allows access to the retaining mechanism to allow disabling of the retaining mechanism.

In a third aspect of the disclosure, there is provided a method of obtaining a sample at a first location, securing the sample for transport and analysing the sample at a second location using the cartridge, the method comprising the following steps:
at the first location, moving the cartridge from the first closed configuration to the first open configuration;
receiving a sample on the porous substrate;
moving the cartridge from the first open configuration to the second closed configuration; and
disabling the retaining mechanism in order to access the porous substrate for analysis.

The method may further comprise, after the step of moving the cartridge from the first open configuration to the second closed configuration, the further step of:
transporting the cartridge to the second location to disable the retaining mechanism in order to access the porous substrate for analysis.

The step of accessing the porous substrate may comprise triggering a tamper evident feature in order to disable the retaining mechanism of the cartridge.

The step of disabling the retaining mechanism and accessing the porous substrate for analysis and then analysing the porous substrate may be performed by a machine.

In a fourth aspect of the disclosure, there is provided an apparatus for use with a mass spectrometer and a cartridge, the apparatus comprising:
an electrical contact for providing an electrical connection to the porous substrate of the cartridge; and
a fluid supply apparatus for spraying the porous substrate;
wherein the apparatus is configured such that the point at the second end of the porous substrate faces an opening of the mass spectrometer though which ions are receivable for analysis.

The apparatus may further comprise:
a release tool configured to release the retaining mechanism so as to release the cartridge from the second closed configuration such that the porous substrate is accessible for analysis in the apparatus.

### BRIEF DESCRIPTION OF THE DRAWINGS

FIG. 1 shows a highly schematic representation of apparatus for paper spray mass spectrometry in accordance with the present disclosure;

FIG. 2 shows a more detailed schematic representation of apparatus for paper spray mass spectrometry configured for fingerprint analysis in accordance with the present disclosure;

FIG. 3 shows a schematic representation of a mass spectrometer inlet relative to a paper spray sample and source in accordance with the present disclosure;

FIG. 4 shows a schematic representation of a regular mass spectrometer suitable for use in paper spray mass spectrometry in accordance with the present disclosure;

FIG. 5 shows a schematic representation of a skin-print sample collection kit suitable for use in paper spray mass spectrometry in accordance with the present disclosure;

FIG. 6 shows an example of mass spectrum derived from a sample containing cocaine;

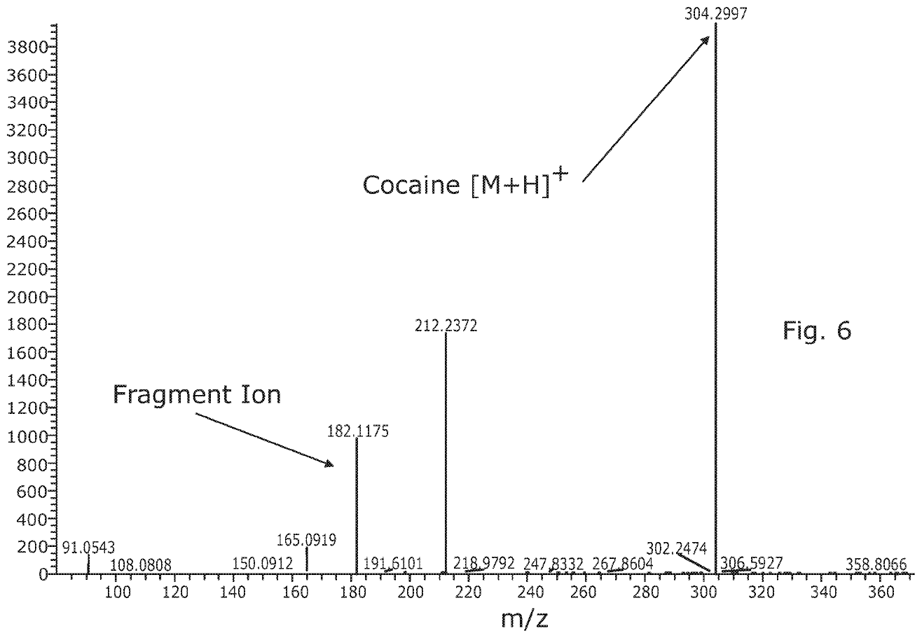

FIG. 7 shows an example of mass spectrum derived from a sample containing a metabolite of cocaine known as benzoylecgonine (BZE);

FIG. 8 shows an example of mass spectrum derived from a sample containing a metabolite of cocaine known as ecgonine methyl ester (EME);

FIG. 9 shows an example of a skin-print developed on a paper spray substrate using silver nitrate as a developing agent;

FIG. 10 shows an example of a skin-print developed on a paper spray substrate using Ninhydrin as a developing agent;

FIG. 11 shows an example MS/MS scanning regime; and

FIG. 12 shows a further example MS/MS scanning regime.

### DETAILED DESCRIPTION

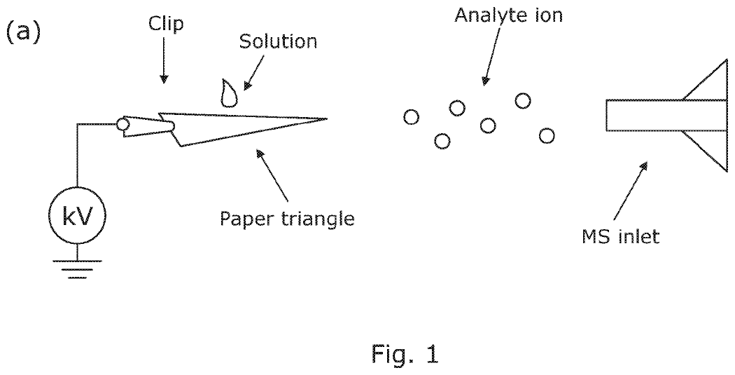

The concept of paper spray mass spectrometry is shown schematically in FIG. 1. The technique involves providing a sample, such as a fingerprint 10, onto a substrate of a porous, wicking material such as paper 20 which is shaped to taper to a point. A solution 30 is applied to the substrate 20. A power supply 40 is used to apply a voltage to the substrate 20 via an appropriate contact such as a metallic clip 50. An electric field arising from the voltage causes gas phase ions 70 to be produced from the surface of the substrate 20. These ionic species, or neutral compounds, are then be detected by a mass spectrometer 60.

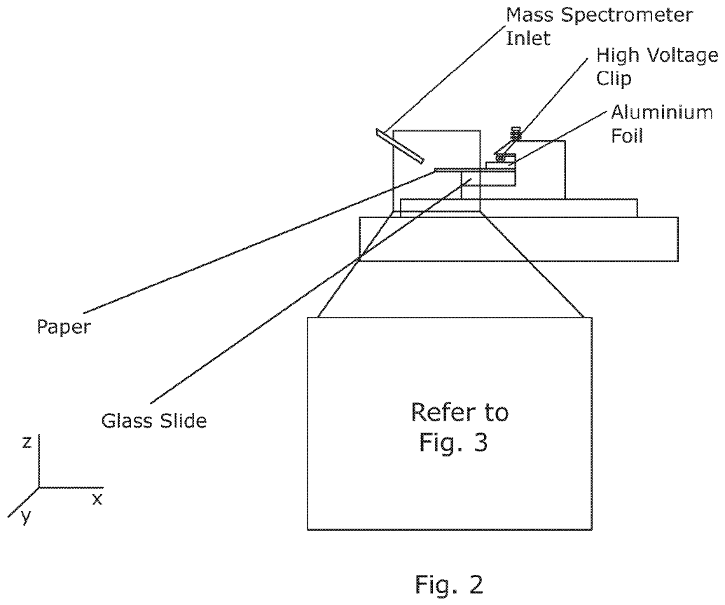

FIG. 2 provides a further schematic representation of the paper spray mass spectrometry technique. In particular, FIG. 2 shows that the substrate 20 may be mounted in a position for analysis by resting on an insulating platform 80, such as a glass slide, and by placing metallic foil 90 (e.g. aluminium foil) between the substrate 20 and the metallic clip 50. In this way, the electric field may be appropriately directed within the substrate.

The substrate 20 may consist of porous fibres such that the solution 30 may migrate randomly through the fibres of the substrate 20 and such that the gas phase ions 70 may escape from the surface of the substrate 20.

The substrate 20 may comprise chromatography paper such as Whatman grade 1 chromatography paper having a thickness of 0.18 mm which results in a flow rate of 130 mm per 30 minutes. Alternatively, the substrate may comprise Whatman Grade 31ET chromatography paper (thickness 0.508 mm, flow rate 225 mm/30 minutes).

Functionalised cellulose paper (ion exchange papers and Whatman DMPK paper) might also be used. In some circumstances it may be beneficial to treat the substrate prior to use.

The substrate 20 may extend in a plane and take a shape of a triangle in the plane. A base 21 of the triangular substrate 20 may be referred to as a first end 23 of the substrate 20 while a vertex 22 of the triangular substrate 20 may be referred to as a second end 24 of the substrate 20.

While the substrate 20 takes the form of a triangle in an exemplary embodiment, the disclosure is not limited to substrates having a strictly triangular form. In particular, while the substrate 20 may have three sides in the plane, some, all or none of these sides may be strictly straight. What is of most importance in relation to the shape of the substrate is that it tapers to a point. The two sides either side of the point need not be straight. There may be one or more sides opposite the point. None of these sides opposite the point needs to be straight.

In an exemplary embodiment, the substrate 20 takes the form of a planar triangle having two sides of equal length wherein the point (vertex 22) is located between two sides of equal length and wherein the triangle has an axis of symmetry through the point (vertex 22).

In a still further refined embodiment the substrate 20 may described in the previous paragraph may be amended simply to round off the corners at the two apexes at either end of the base, opposite the vertex 22. In this way, flow of ions towards the two apexes at either end of the base may be attenuated in favour of flow of ions towards the vertex 22.

In an exemplary embodiment, the substrate 20 is of Whatman grade 1 chromatography paper having a thickness of 0.18 mm. The shape of the substrate 20 in the plane is substantially an isosceles triangle having a base of 1.6 cm and a height from base to vertex of 2.1 cm, thus providing a substrate having a surface area of 1.68 cm². In some arrangements, the corners of the two apexes at either of the end of the base may be rounded off, thus slightly reducing the area of the substrate.

The substrate 20 (once the sample has been applied thereto) may be held in position for analysis between the insulating platform 80 and the metallic foil 90 such that the base 21 of the triangular substrate 20 may be retained in position between the metallic foil 90 and the insulating platform 80 and such that a vertex 22 of the triangular substrate 20 may project freely from the insulating platform 80 and the metallic foil 90. In this way, a first end 23 of the substrate 20 may be said to be retained while a second end 24 of the substrate may be said to be freely projecting.

An inlet 65 of the mass spectrometer 60 may be located adjacent the vertex 22 of the triangular substrate 20.

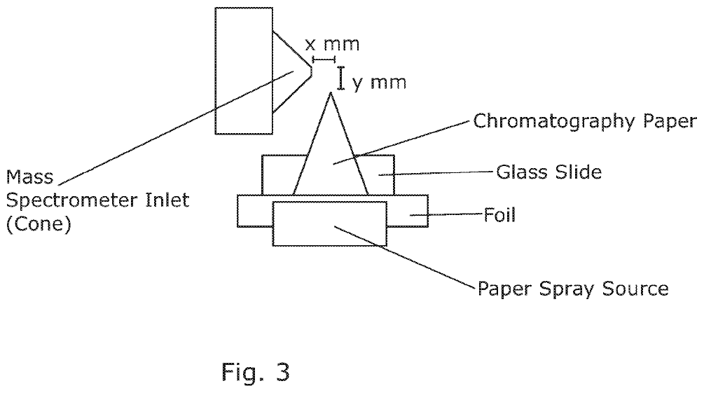

FIG. 3 shows a schematic representation of a spatial arrangement the triangular substrate 20 relative to the mass spectrometer inlet 65. As shown, the mass spectrometer inlet 65 may comprise a frusto-conical aperture 66. An axis of the frusto-conical aperture 66 may intersect an axis of that intersects with the vertex 22 of the triangular substrate 20 and bisects the base 21 of the triangular substrate 20. The axis of the frusto-conical aperture 66 and the axis through the vertex 22 of the triangular substrate 20 may intersect at a distance x from the frusto-conical aperture 66 and at a distance y from the vertex 22 of the triangular substrate 20.

In this way, the frusto-conical aperture 66 mass spectrometer inlet 65 may be substantially perpendicular to a direction of ions that may be emitted from the vertex 22 of the triangular substrate 20 and at an appropriate distance from the location of the vertex 22 of the triangular substrate 20 at which the ions leave the triangular substrate 20.

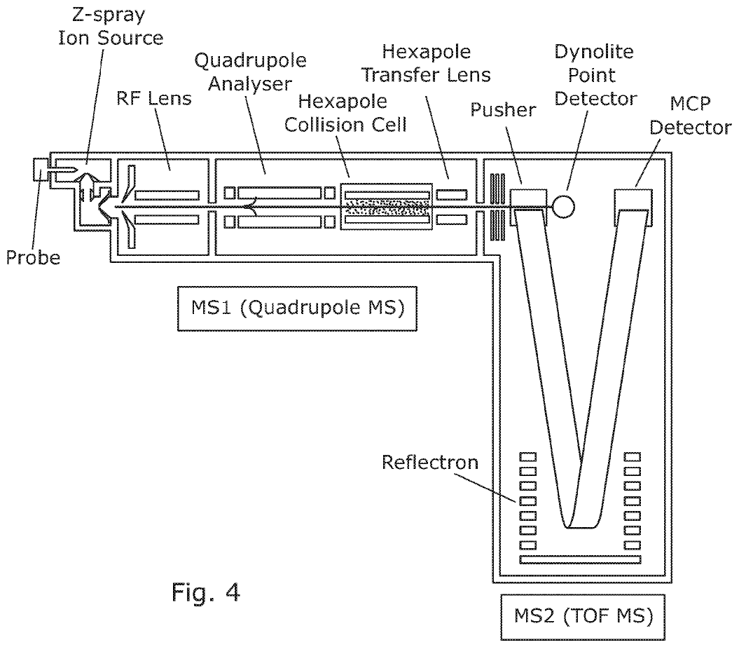

FIG. 4 shows a schematic representation of a mass spectrometer 400 that might be used in the context of the analysis of the present disclosure. The mass spectrometer 400 may comprise an inlet 65, a lens 410, a quadrupole analyser 420, a hexapole collision cell 430, a hexapole transfer lens 440 a pusher 450, a dynolie point detector 460, an ion mirror 470, and a MCP detector 480. In this way, the spectrometer 400 may comprise both a quadrupole mass spectrometer and a time of flight mass spectrometer, abbreviated to a QTiF mass spectrometer.

As the skilled person readily appreciates, the present disclosure is not limited to a mass spectrometer 400 of the type shown in FIG. 4. A wide range of mass spectrometers is available commercially and/or for bespoke design in the context of the present disclosure. The disclosure is not to be understood as being limited to the example mass spectrometer shown schematically in FIG. 4.

Various mass spectrometry techniques may be employed. One technique that may be particularly appropriate in the context of the present disclosure may be MS/MS analysis. Alternatively, an Orbitrap mass spectrometer may be used.

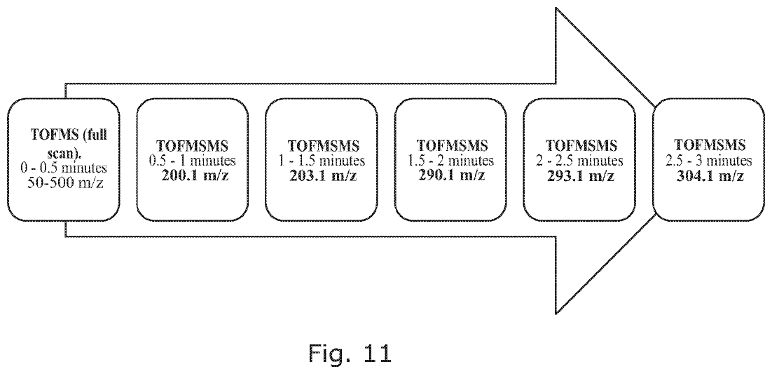

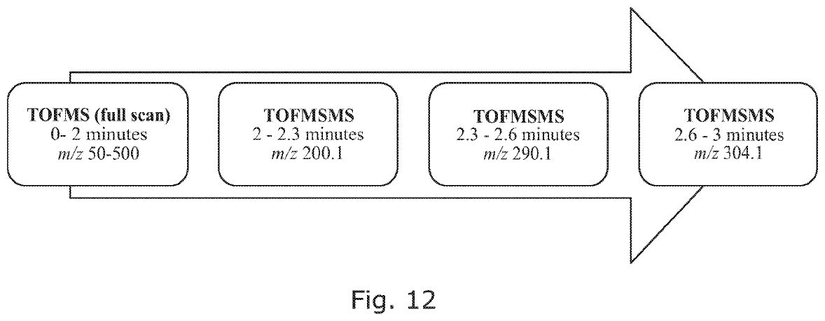

MS/MS analysis may involve a plurality of individual mass spectrometry functions. For example, it may comprise 6 functions, each lasting for 30 seconds. Function 1 may comprise a full scan measurement over a m/z range of 50 to 500. Functions 2 to 6 may comprise tandem MS measurements for each of the analytes of interest. Collision induced dissociation (CID) may be used for fragmentation of the parent ion. Collision energy may be set to 25 eV. Example scanning regimes are illustrated in FIGS. 11 and 12.

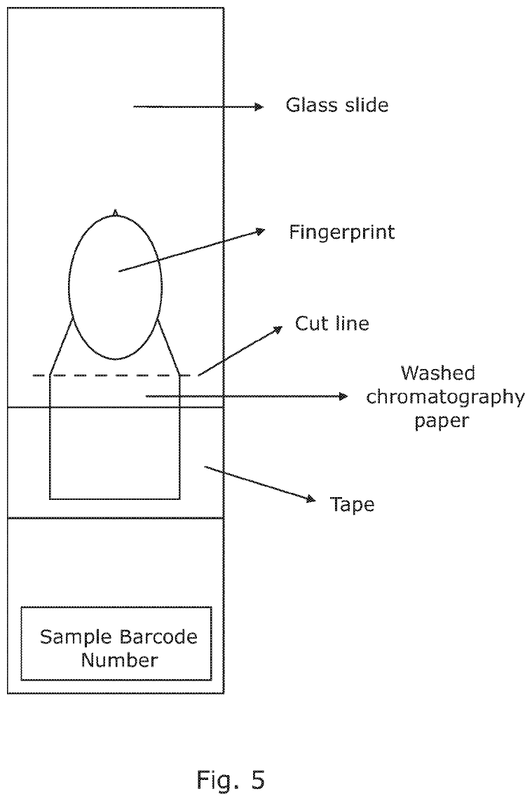

A skin-print sample 10 for analysis using the technique of the present disclosure may be collected using a collection kit 500 of the kind illustrated in FIG. 5. The collection kit 500 may comprise a rigid, planar tile 510, such as a glass slide 510. The collection kit 500 may also comprise an extended substrate 510 which includes the substrate 20 discussed previously in relation to FIGS. 1, 2 and 3 and also an extension portion 530 via which the extended substrate 520 may be fastened to the rigid, planar tile 510. Means of fastening the extended substrate 520 to the rigid, planar tile 510 may include but are not limited to adhesive tape 540. The substrate 20 may be detached from the extension portion 530 by cutting along a cut line 550. The cut line may be pre-weakened, e.g. perforated, to form a frangible locus 550. The frangible locus 550 may be straight or curved in the plane.

The collection kit 500 thus allows a skin-print sample 10, such as a fingerprint sample 10, to be obtained by a user pressing the skin-print 10 onto the planar tile 510 such that the skin-print is received at least in part onto the substrate 20. A remainder of the skin-print may be received onto the rigid, planar tile.

The substrate 20 may be separated from the extended substrate 520 once the skin-print has been deposited. In this way the substrate 20 may be analysed using the paper spray mass spectrometry technique of the present disclosure by fastening the substrate 20 between the insulating platform 80 and the metallic foil 90 (as shown in FIG. 2) such that a vertex 22 of the substrate 20 projects freely adjacent the mass spectrometer inlet 65 (as shown in FIG. 3).

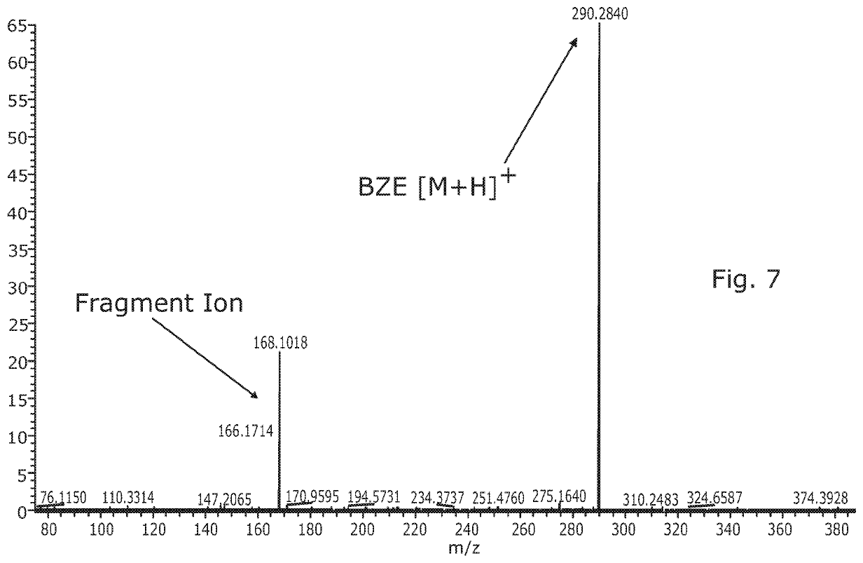

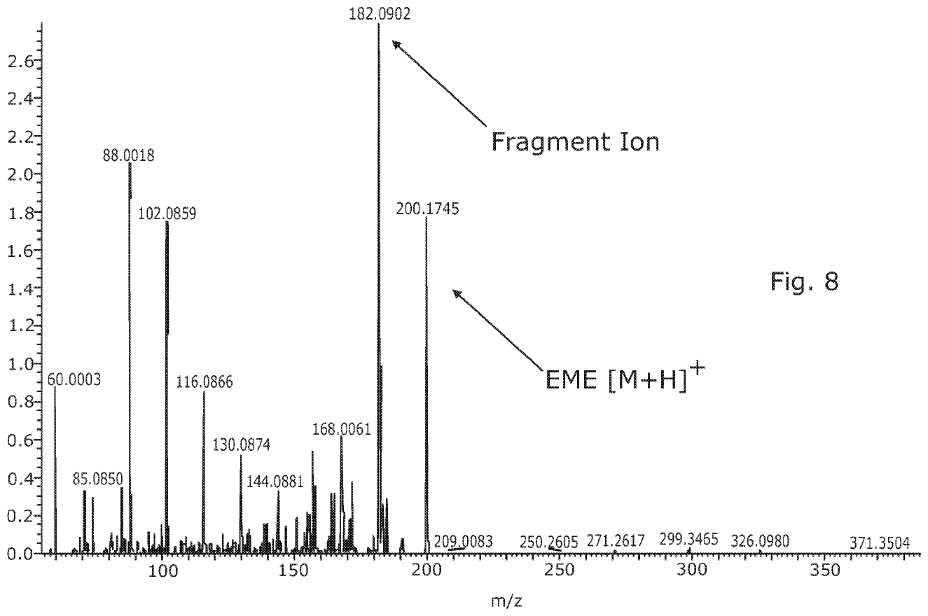

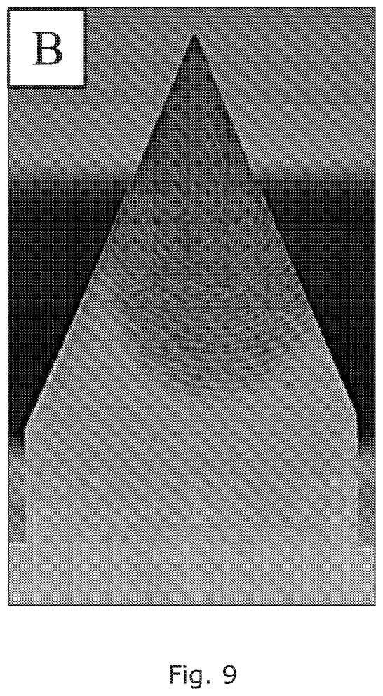

FIGS. 7, 8 and 9 show mass spectra obtained using the technique of the present disclosure. FIG. 7 shows a mass spectrum obtained from a sample containing cocaine. FIG. 8 shows a mass spectrum obtained from a sample containing a metabolite of cocaine known as benzoylecgonine (BZE). FIG. 9 shows a mass spectrum obtained from a sample containing a metabolite of cocaine known as ecgonine methyl ester (EME).

The technique is not limited to the detection of any particular analyte, such as any particular chemical species (e.g. cocaine) or any particular metabolite thereof (e.g. BZE and/or EME). Rather, the technique may be used to screen for any analyte of interest which may or may not be a narcotic, an opiate or a metabolite thereof. Amino acids may also be detected by the technique.

The process for obtaining the sample and conducting the analysis may be performed as follows.

The extended substrate 520 may be pre-treated, for example by immersing in a solution of hydrochloric acid (HCl) (for example 0.1% HCl) followed by a methanol/water rinse (for example 50:50 MeOH:H₂O) and allowed to air dry. This may reduce background signal measured by the mass spectrometer.

The skin-print sample may then be provided on the extended substrate 520 in a region of the vertex 22 of an upper surface of the substrate 20 (FIG. 5). The substrate 20 may be detached from the remainder of the extended substrate 520 and placed with its lower surface (opposite the upper surface) resting on the insulating platform 80 (FIGS. 2 and 3) such that the upper surface of the substrate 20 faces upwards. Metallic foil 90 may be applied to a region of the base 21 of the upper surface of the substrate 20. The metallic clip 50 may be applied to the metallic foil 90, with the metallic clip connected to the power supply 40. The vertex 22 of the substrate 20 may project freely.

The orientation of the substrate relative to the may be as described in relation to the mass spectrometer inlet 65 may be as described above in relation to FIG. 3.

Next, the substrate 20 containing a skin-print may be wetted with a spray solvent. In an exemplary embodiment, the solvent applied to the substrate 20 may comprise or consist of 70:30 MeOH:H₂O or 80:20 MeOH:H₂O. The volume of solvent may comprise or consist of 60 μl or 80 μl or 100 μl.

A voltage of at least 2 kV and preferably 3.5 kV or 4 kV or 4.5 kV may be applied to the metallic clip 50. At the same time, the acquisition of ions through the mass spectrometer inlet may begin. This may be in full scan mode so as to detect mass to charge over a ratio of 50 to 500 m/z. The voltage application and the acquisition time may be for a duration of 2 minutes. The voltage may then be removed.

Subsequently, a further volume of spray solvent may be added, as previously. The voltage may then be reapplied for a further period of, for example, 2 minutes, during which MS/MS measurements may be obtained.

Analysis of the spectra obtained in accordance with the mass spectroscopy techniques may be performed in accordance with techniques known in the art.

Optical development of friction ridges of the skin-print for the purposes of skin-print identification is also within the scope of the disclosure. Techniques have been developed to enable development of optically obtainable (e.g. by optical digital photography) in such a manner as to enable subsequent analysis by paper spray mass spectroscopy have been developed and are now discussed.

In one embodiment, silver nitrate is used as part of a technique for skin-print visualisation that is compatible with paper spray mass spectrometry. In short, the process relies upon reaction of silver ions in the silver nitrate with chlorides in a skin-print to form silver chloride that causes colouration. Chlorides in the skin-prints are most concentrated in the ridge portions of the impression of the skin-print on the substrate and so these portions result in the greatest colouration. The pattern of the resultant colouration reflects the pattern of the skin-print and thus assists in visualisation.

In one arrangement, a silver nitrate solution (between 0.010 M and 0.05 M, preferably) is deposited onto a paper spray substrate and allowed to dry prior to skin-print collection. The volume of silver nitrate solution may be between 20 μl and 60 μl, such as 40 μl. A particularly preferred embodiment uses 60 μl of 0.015 M. Next, a skin-print is deposited on the paper spray substrate. A mass of the skin-print deposition on the substrate may be measured on weighing scales. A measurement of between 100 g and 200 g may be appropriate. Then the substrate is exposed to ultra-violet light for a period of between 5 and 10 minutes, preferably 5 minutes. This results in the skin-print being visually developed and suitable for optical capture and analysis. An example of a skin-print sample developed using silver nitrate as described is shown in FIG. 9.

In an alternative, ninydrin may be used to develop a skin-print on a paper spray substrate. Ninydrin reacts with primary amine groups to form a purple colouration. In skin-prints, ninydrin reacts with naturally secreted amino acids which results in purple colouration of the skin-print ridges, enabling visualisation of the skin-print pattern. This must be achieved in such a way as not to affect the detection of any of the analytes of interest.

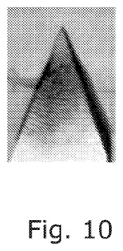

In one embodiment, a 5 mg/ml ninhydrin solution is 95:5 absolute ethanol:acetone is applied using a spray bottle over the skin-print sample deposited on the substrate. Samples are left in ambient conditions for one hour to allow development time. Skin-print details are visible in purple as shown in FIG. 10.

1. A method of analysing a skin-print comprising the steps of:
providing a porous substrate, the porous substrate extending in a substrate plane and being substantially planar and having a first end and a second end, wherein the second end tapers to a point;
applying to the porous substrate a skin-print to be analysed;
applying a fluid to the porous substrate;
applying a voltage between the first end of the porous substrate and ground to generate an electric field by which droplets of the fluid are ionised and emitted from the point in the form of a Taylor cone;
analysing the ionised droplets using a mass spectrometer.

2. The method of claim 1 wherein the porous substrate is substantially triangular.

3. The method of claim 2 wherein the first end forms a base of the triangular porous substrate and the second end forms an apex of the triangular porous substrate.

4. The method of any preceding claim wherein the mass spectrometer comprises a mass spectrometer inlet having an aperture and an inlet axis substantially perpendicular to the aperture.

5. The method of claim 4 wherein the mass spectrometer inlet is located such that the inlet axis is substantially perpendicular to the point of the porous substrate.

6. The method of claim 4 or claim 5 wherein mass spectrometer inlet is located such that the inlet axis is substantially in the substrate plane.

7. The method of any of claims 4 to 6 wherein the mass spectrometer inlet comprises a frusto-conical aperture wherein the inlet axis is substantially coincident with the axis of the frusto-conical aperture.

8. The method of any preceding claim wherein the area of the porous substrate is between 0.2 cm² and 3.0 cm², preferably between 1.0 cm² and 2.0 cm², more preferably between 1.5 cm² and 1.9 cm², still more preferably between 1.6 cm² and 1.7 cm², most preferably approximately 1.68 cm².

9. The method of any preceding claim wherein the voltage is between 2 kV and 10 kV, preferably between 3.5 kV and 5.5 kV, and more preferably, between 4.4 kV and 4.6 kV, most preferably 4.5 kV.

10. The method of any preceding claim wherein the step of applying the fluid to the porous substrate comprises applying a volume of fluid of between 10 μl and 1,000 μl, preferably between 50 μl and 200 μl, more preferably between 70 μl and 130 μl, still more preferably between 90 μl and 110 μl, and most preferably approximately 100 μl.

11. The method of any preceding claim further comprising the step of:
performing an optical analysis of the skin-print prior to applying the fluid to the porous substrate.

12. The method of claim 11 wherein the step of performing the optical analysis comprises performing a colourmetric analysis of the skinprint.

13. The method of any preceding claim further comprising the step of:
applying a developing agent to the porous substrate including the skin-print to be analysed in order to develop the skinprint prior to applying the fluid to the porous substrate.

14. The method of claim 13 wherein the developing agent is or comprises Ninhydrin.

15. The method of claim 13 wherein the developing agent is or comprises silver nitrate.

16. The method of claim 14 or claim 15 further comprising a step of developing the developing agent by application of electromagnetic radiation.

17. The method of claim 16 wherein the step of developing the developing agent comprises exposing the developing agent to ultra-violet light.

18. The method of claim 17 wherein the ultra-violet light has a wavelength of approximately 254 nm.

19. The method of claim 17 or 18 wherein the step of exposing the developing agent to ultra-violet light is conducted for a period of 1 to 15 minutes, preferably 2 to 10 minutes, more preferably 3 to 7 minutes, even more preferably 4 to 6 minutes and most preferably approximately 5 minutes.

20. The method of any of claims 13 to 19 when dependent upon claim 11 or claim 12, wherein the optical analysis is performed on the skin-print following application of the developing agent.

21. The method of any preceding claim wherein the porous substrate is treated with silver nitrate prior to applying the skin-print to be analysed to the porous substrate.

22. A cartridge comprising a housing and a porous substrate located within the housing, the porous substrate being substantially planar and having a first end and a second end, wherein the second end tapers to a point.

23. The cartridge of claim 22 further comprising a first electrical contact connected to a part of the porous substrate distant from the point and a second electrical contact on an exterior of the housing electrically connected to the first electrical contact.

24. The cartridge of claim 22 or claim 23 further comprising a shutter openable to provide access to the porous substrate and closable to restrict access to the porous substrate.

25. The cartridge of any of claims 22 to 24 wherein the housing comprises at least a first part and a second part which are movable relative to one another
i) from a first closed configuration in which the substrate chamber is inaccessible;
ii) to a first open configuration in which access to the substrate chamber is enabled to allow capturing of a skin-print on a sample-capture substrate contained within the substrate chamber; and subsequently
iii) into a second closed configuration in which the substrate chamber is again inaccessible;
wherein the cartridge further comprises a retaining mechanism for retaining the cartridge in the second closed configuration wherein the retaining mechanism is disablable to permit movement of the cartridge out of the second closed configuration
wherein the cartridge further comprises a tamper evident feature that prevents access to the retaining mechanism,
wherein access to the retaining mechanism for disabling the retaining mechanism is available only by triggering the tamper evident feature. a first closed position,

26. The cartridge of claim 25 wherein access to the retaining mechanism for disabling the retaining mechanism is restricted.

27. The cartridge of claim 25 or claim 26 further comprising a non-return feature which prevents movement from the first open configuration to the first closed configuration.

28. The cartridge of any of claims 25 to 27 further comprising a second open configuration accessible only by disabling the retaining mechanism.

29. The cartridge of any of claims 25 to 28 wherein the tamper evident feature comprises a frangible element having an undamaged initial configuration in which the retaining mechanism is inaccessible and a damaged configuration which allows access to the retaining mechanism to allow disabling of the retaining mechanism.

30. A method of obtaining a sample at a first location, securing the sample for transport and analysing the sample at a second location using the cartridge of any of claims 25 to 29, the method comprising the following steps:
at the first location, moving the cartridge from the first closed configuration to the first open configuration;
receiving a sample on the porous substrate;
moving the cartridge from the first open configuration to the second closed configuration; and
disabling the retaining mechanism in order to access the porous substrate for analysis in accordance with any of claims 1 to 21.

31. The method of claim 30 further comprising, after the step of moving the cartridge from the first open configuration to the second closed configuration, the further step of:
transporting the cartridge to the second location to disable the retaining mechanism in order to access the porous substrate for analysis.

32. The method of claim 30 or claim 31 wherein the step of accessing the porous substrate comprises triggering a tamper evident feature in order to disable the retaining mechanism of the cartridge.

33. The method of any of claims 30 to 32 wherein the step of disabling the retaining mechanism and accessing the porous substrate for analysis and then analysing the porous substrate is performed by a machine.

34. An apparatus for use with a mass spectrometer and a cartridge of any of claims 25 to 29, the apparatus comprising:
an electrical contact for providing an electrical connection to the porous substrate of the cartridge; and
a fluid supply apparatus for spraying the porous substrate;
wherein the apparatus is configured such that the point at the second end of the porous substrate faces an opening of the mass spectrometer though which ions are receivable for analysis.

35. The apparatus of claim 34 further comprising:
a release tool configured to release the retaining mechanism so as to release the cartridge from the second closed configuration such that the porous substrate is accessible for analysis in the apparatus.
